(19) United States
(12) Patent Application Publication
(10) Pub. No.: US 2005/0281377 A1
(43) Pub. Date: Dec. 22, 2005

(54) X-ray apparatus with an ultra-capacitor for storing energy

(76) Inventor: Udo Heinze, Erlangen (DE)

Correspondence Address: SCHIFF HARDIN LLP;Patent Department, 6600 Sears Tower, 233 South Wacker Drive, Chicago, IL, 60606, US

(21) Appl. No.: 10/996,940

(22) Filed: Nov. 24, 2004

(30) Foreign Application Priority Data
Nov. 27, 2003 (DE) 103 55 424.6

(51) Int. Cl. H05G 1/10; A61B 6/00

(52) U.S. Cl. 378/101

(57) ABSTRACT

An x-ray apparatus that can be connected to an electric mains and has an additional unit to store energy, including at least one ultra-capacitor.

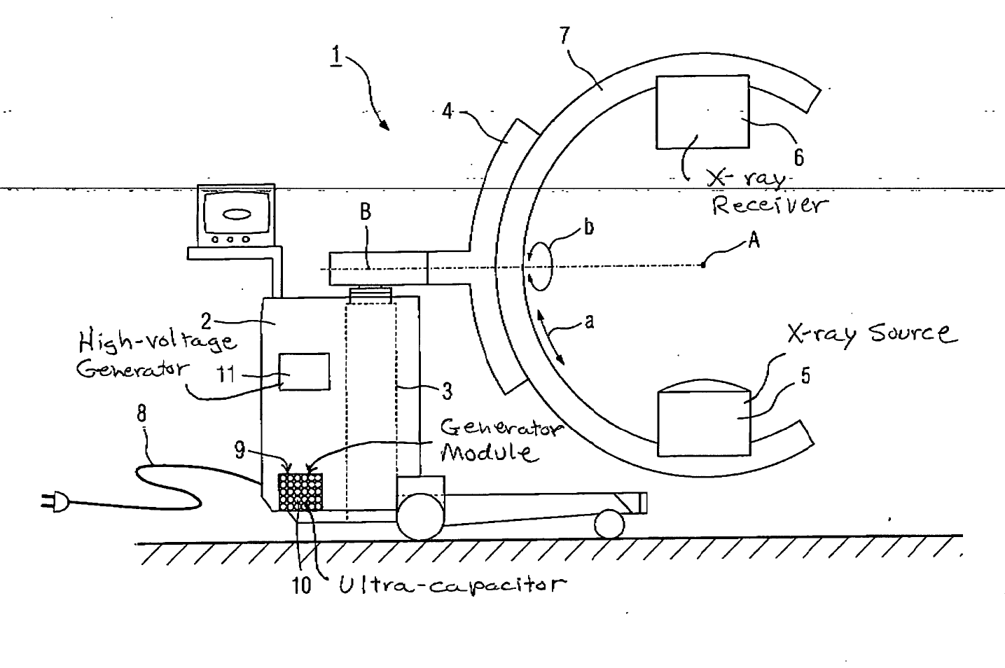

### BACKGROUND OF THE INVENTION

1. Field of the Invention

The present invention concerns an x-ray apparatus which can be connected with an electric mains for supply with electrical energy and that has additional means to store energy.

2. Description of the Prior Art

The energy feed available from an electric mains, in particular for the generation of x-ray radiation with an x-ray source of an x-ray apparatus, is limited. Thus, for example, a mains capacity of approximately 2500 Watts maximum is available from a single-pole mains connection for a mobile C-arm x-ray apparatus. Since x-ray apparatuses can be operated in modes in which this maximum power that can be drawn from a mains connection can be temporarily exceeded, present x-ray apparatuses, for example the POWERMOBIL from Siemens AG, have additional capacitors for energy storage for a short-term energy boost or augmentation to provide sufficient energy to the components of the x-ray apparatus in the aforementioned operating modes. Such an x-ray apparatus is described in German PS 197 49 944 in which, to prevent a failure in an operating mode in which the x-ray apparatus has an increased energy requirement, the x-ray generator is connected to an electric mains via capacitors serving for energy boosting.

Furthermore, it is known in mobile x-ray apparatuses to use lead accumulators lead storage batteries for a longer storage of energy in order to be able to operate independently of a mains connection, at least for a certain amount of time. Lead accumulators, however, have the disadvantage of being relatively heavy, voluminous, less environmentally friendly and maintenance-intensive.

### SUMMARY

An object of the present invention is to execute and provide an x-ray apparatus of the initially described type, wherein the means for energy storage occupies a relatively small overall volume in the x-ray apparatus as well as being light and maintenance-friendly.

This object is achieved in accordance with the invention by an x-ray apparatus that can be connected with an electric mains for supply with electrical energy and that has at least one ultra-capacitor serving as an additional energy source, namely a stored energy source. A number of ultra-capacitors can be provided to store energy in the x-ray apparatus. Ultra-capacitors are a type of capacitor also known as “Electrochemical Double Layer (ECDL), capacitors” and “super-capacitors”. Ultra-capacitors store energy electrostatically, by polarization of an electrolytic solution. Such ultra-capacitors are available, for example, from the company Maxwell Technologies under the trademark BOOSTCAP® Ultracapacitor. Ultra-capacitors meet the requirement to store electrical energy practically without maintenance in an x-ray apparatus under operational demands of (in comparison to lead accumulators) a small overall volume and low weight, such that less space must be provided in the x-ray apparatus for the storage of energy. As a result, the x-ray apparatus is lighter overall and lower operating costs occur in the use of the x-ray apparatus due to the freedom from maintenance.

In a preferred embodiment of the invention, a generator module for energy storage has a number of ultra-capacitors in order to be able to store energy necessary for the operation of the x-ray apparatus and to provide it as needed. The energy stored In the generator module can be used to temporarily operate the x-ray apparatus with only the energy that can be removed from the generator module, or it can be used as an additional energy source when the x-ray apparatus is operated in a mode in which the energy that can be drawn from an available mains connection is insufficient for the failure-free operation of the x-ray apparatus. The possibility exists to permanently reduce the connection power of the x-ray apparatus and to draw the energy additionally required for the operation of the x-ray apparatus from the generator module. The generator module is then preferably charged during the x-ray radiation pauses of the x-ray apparatus.

In a further embodiment of the invention the x-ray apparatus is a mobile C-arm x-ray apparatus, with an x-ray source and an x-ray receiver mounted opposite one another on the C-arm.

### DESCRIPTION OF THE DRAWING

The single FIGURE is a schematic illustration of an x-ray apparatus constructed in accordance with the principles of the present invention.

### DESCRIPTION OF THE PREFERRED EMBODIMENTS

In an exemplary embodiment, the inventive x-ray apparatus is a C-arm x-ray apparatus 1 with a mobile apparatus cart 2 movable on wheels. The C-arm x-ray apparatus 1 has a lifting device 3 connected to a support part 4 is connection. A C-arm 7 has an x-ray source 5 and an x-ray receiver 6 mounted thereon and is supported on the support part 4. The C-arm 7 is adjustable isocentrically along its circumference around the orbital axis A in the exemplary embodiment, as indicated by the double arrow. Together with the support part 4, the C-arm 7 can be pivoted on its angulation axis B in the directions of the double arrow b.

To supply the C-arm x-ray apparatus 1 with electrical energy, the C-arm x-ray apparatus 1 has a network cable 8 which can be connected to a mains outlet (not shown, but known). The mains connection is normally a typical household one-pole mains connection. Furthermore, the C-arm x-ray apparatus 1 comprises on a schematically indicated manner) a generator module 9 containing a number of ultra-capacitors 10. The generator module 9 with ultra-capacitors 10 serves for the storage of energy, so that, at least temporarily, a mains-independent operation of the C-arm x-ray apparatus I is enabled. Moreover, it enables the generator module 9 to provide the C-arm x-ray apparatus 1 with additional energy in operating modes of the C-arm x-ray apparatus 1 in which more energy is required than can be drawn from an available mains connection via the mains cable 8. Furthermore, via the generator module 9 it is possible to permanently reduce the network connection power, for example to less than 1.5 kW. and to provide the remainder of the energy necessary for the operation of the C-arm x-ray apparatus 1 via the generator module 9.

The generator module 9 is, insofar as the connection exists, normally continuously charged from the energy supply grid, The generator module 9 is primarily charged during the x-ray radiation pauses of the C-arm x-ray apparatus 1. For this, the generator module 9 is connected (in a manner not shown) with the mains able 8 for energy supply.

As an example for a device component to be supplied with electrical energy, in the FIGURE a high-voltage an x-ray generator 11 is shown connected (in a manner not shown) with the mains cable 8 and the generator module 9. The high-voltage generator 11 feeds the x-ray source 5 via an electrical connection (likewise not shown).

The invention has been specified above as an example of a mobile C-arm x-ray apparatus, however, the use of the invention is not limited to a mobile C-arm x-ray apparatus.

Although modifications and changes may be suggested by those skilled in the art, it is the intention of the inventor to embody within the patent warranted hereon all changes and modifications as reasonably and properly come within the scope of his contribution to the art.

1. An x-ray apparatus composing:
a plurality of power-consuming components;
a connection to an electric mains for supplying electrical power to said power-consuming components; and
an energy storage unit connected to said power-consuming components for supplying power to said power-consuming components separate from the power supplied to said power-consuming, components via said connection to said electric mains, said energy storage unit comprising at least one ultra-capacitor.

2. An x-ray apparatus as claimed in claim 1 wherein said energy storage unit comprises a generator module containing a plurality of ultra-capacitors.

3. An x-ray apparatus as claimed in claim 1 comprising a mobile cart on which said plurality of power-consuming components are mounted.

4. An x-ray apparatus as claimed in claim I comprising a C-arm. and wherein said power-consuming components include an x-ray source and a radiation receiver mounted opposite each other on said C-arm.
